(19) United States
(12) Patent Application Publication
(10) Pub. No.: US 2022/0084672 A1
(43) Pub. Date: Mar. 17, 2022

(54) TEMPERATURE MONITORING WEARABLE WITH AN ARTIFICIAL INTELLIGENCE ENGINE

(71) Applicant: SportsMEDIA Technology Corporation, Durham, NC (US)

(72) Inventor: Gerard J. Hall, Durham, NC (US)

(73) Assignee: SportsMEDIA Technology Corporation, Durham, NC (US)

(21) Appl. No.: 17/478,322

(22) Filed: Sep. 17, 2021

Related U.S. Application Data
(60) Provisional application No. 63/079,752, filed on Sep. 17, 2020.

(51) Int. Cl. G16H 40/67; G16H 10/60; G16H 50/20; G16H 15/00; A61B 5/00; A61B 5/0205; A61B 5/024; A61B 5/021; A61B 5/145; A61B 5/1455

(52) U.S. Cl. CPC G16H 40/67; A61B 5/4806; G16H 50/20; G16H 15/00; A61B 5/6824; A61B 5/6831; A61B 5/7275; A61B 5/02055; A61B 5/02438; A61B 5/021; A61B 5/14546; A61B 5/14551; A61B 5/002; A61B 5/0022; A61B 5/7475; A61B 5/743; A61B 5/746; G16H 10/60

(57) ABSTRACT

The present invention includes a sleep system with a temperature monitoring wearable that is operable to capture biometric and physiological data of a user while the user is sleeping. The present invention further includes an artificial intelligence engine that is operable to receive and analyze the captured biometric and physiological data.

### CROSS REFERENCES TO RELATED APPLICATIONS

This application is related to and claims priority from the following US patents and patent applications: it claims priority to and the benefit of U.S. Provisional Application No. 63/079,752, filed on Sep. 17, 2020, which is incorporated herein by reference in its entirety.

### BACKGROUND OF THE INVENTION

### 1. Field of the Invention

The present invention relates to collecting and analyzing biometric data and more specifically to using wearable devices and artificial intelligence to collect and analyze real-time or near real-time biometric data.

### 2. Description of the Prior Art

It is generally known in the prior art to provide to collect biometric data using a temperature sensor.

Prior art patent documents include the following:

US Patent Publication No. 2014/0316220 for Personal health monitoring system by inventor Sheldon, filed Jul. 1, 2014 and published Oct. 23, 2014, discloses a personal health monitor device including a memory for collecting and storing attributes from an individual and a processor for quantizing each attribute in such a way as to indicate a normal range for that attribute and for measuring deviations from that normal range. The processor further calculates the well-being of the individual using the deviations measured. The results are displayed indicating the well-being of the individual.

U.S. Pat. No. 9,603,524 for Portable biometric monitoring devices and methods of operating same by inventors Park et al., filed Nov. 6, 2013 and issued Mar. 28, 2017, discloses a portable biometric monitoring device including a housing having a physical size and shape that is adapted to couple to the user's body, at least one band to secure the monitoring device to the user, a physiological sensor, disposed in the housing, to generate data which is representative of a physiological condition of the user data. The physiological sensor may include a light source to generate and output light having at least a first wavelength, and a photodetector to detect scattered light (e.g., from the user). A light pipe is disposed in the housing and optically coupled to the light source directs/transmits light therefrom along a predetermined path to an outer surface of the housing. Processing circuitry calculates a heart rate of the user using data which is representative of the scattered light.

US Patent Publication No. 2014/0257058 for Automated personal medical diagnostic system, method, and arrangement by inventors Clarysse et al., filed Oct. 19, 2012 and published Sep. 11, 2014, discloses an automated personal medical diagnostic system and arrangement, including: at least one sensor configured to measure and/or sense at least one physiological condition and generate or acquire sensor data; at least one computing device configured to process at least a portion of the sensor data and generate diagnostic data based at least partially on the sensor data; and at least one user interface configured for user interaction; wherein the diagnostic data at least partially comprises at least one of the following: indicator data, medical diagnostic data, trigger data, or any combination thereof. A method for automated medical diagnosis is also disclosed.

U.S. Pat. No. 7,004,910 for System and method for monitoring body temperature by inventor Lindsey, filed Dec. 12, 2002 and issued Feb. 28, 2006, discloses a system comprising one or more sensor devices, where each sensor device is capable of measuring a temperature at a known location of a body of a human or other animal. Each sensor transmits a temperature measurement value and sensor identification to a monitor device. The monitor device receives the temperature measurement value and sensor identification, and computes an adjusted temperature value based upon the position of the measuring sensor, body age, and time of day. The adjusted temperature value is tested for alarm conditions and displayed for use by a caretaker.

U.S. Pat. No. 10,410,308 for System, method, and device for personal medical care, intelligent analysis, and diagnosis by inventors Abousy et al., filed Apr. 16, 2007 and issued Sep. 10, 2019, discloses a system for personal medical care, intelligent analysis and diagnosis including: at least one source of medical information; at least one source of personal medical data for at least one patient; and one or more servers, where the medical information and the personal medical data are accessible to the server(s). The server(s) may include: an artificial intelligence (AI) component for analyzing the personal medical data with the medical information and identifying at least one issue requiring follow-up by the patient or by at least one external authorized entity; and at least one real-time communication link for bi-directional communication with at least one external authorized entity.

US Patent Publication No. 2014/0343389 for Wireless monitoring device by inventors Goldstein et al., filed May 20, 2014 and published Nov. 20, 2014, discloses a monitor and monitoring system suitable for attachment to the skin of a mammal, including a human. The monitor and monitoring system are designed for continuous wireless real-time measurement of physiological signals and transmission of the measurements to a remote computer or mobile device.

US Patent Publication No. 2010/0052914 for Portable body temperature continuous monitoring system by inventor Tsai, filed Aug. 28, 2008 and published Mar. 4, 2010, discloses a portable body temperature continuous monitoring system includes a wearable thermal detecting device, a recording device, a displaying device and an alarming device. The recording device records and stores a human body temperature detected by the wearable thermal detecting device, and then transmits a temperature signal to the displaying device and the alarming device through a wireless method. Thereby, remote and continuous monitoring of a patient's body temperature through the displaying device is possible. Besides, since the system is capable of communicating outward through the alarming device, the patient's abnormal body temperature can be reported to others so that proper treatment can be conducted timely.

US Patent Publication No. 2006/0235328 for Apparatus and method to monitor body temperature by inventor Willis, filed Apr. 19, 2006 and issued Nov. 11, 2008, discloses an apparatus made of a garment having at least one connector to receive an electronic transmission module and an electronic monitor configured to remotely receive and control electronic transmission from the electronic transmission module. The garment includes a sensor to detect the temperature of the wearer. This invention also provides a connector for making an electrical connection. This invention also provides a method for monitoring the body temperature of the wearer.

U.S. Pat. No. 9,204,806 for Apparatus using temperature data to make predictions about an individual filed by inventors Stivoric et al., filed Oct. 30, 2007 and issued Dec. 8, 2015, discloses systems, methods, and devices capable of deriving and predicting the occurrence of a number of physiological and conditional states and events based on sensed data. The systems, methods, and devices utilize the predicted and derived states for a number of health and wellness related applications including the administering therapy and providing actionable data for lifestyle and health improvement.

U.S. Pat. No. 10,561,362 for Sleep assessment using a home sleep system by inventors Reich et al., filed Sep. 16, 2016 and issued Feb. 18, 2020, discloses a sleep assessment device including biometric sensors for detecting a heart rate, respiration, or movement of a user. The device detect a user's sleep state by reading signals from the biometric sensor and logs detected information in a sleep record. The device then compares the logged information to a thumbprint of sleep patterns indicative of sleep disorders and outputs an indication of potential sleep disorders based on the comparison.

U.S. Pat. No. 9,459,597 for Method and apparatus to provide an improved sleep experience by selecting an optimal next sleep state for a user by inventors Kahn et al., filed Feb. 28, 2013 and issued Oct. 4, 2016, discloses a sleep sensing system comprising a sensor to obtain real-time information about a user, a sleep state logic to determine the user's current sleep state based on the real-time information. The system further comprising a sleep stage selector to select an optimal next sleep state for the user, and a sound output system to output sounds to guide the user from the current sleep state to the optimal next sleep state.

### SUMMARY OF THE INVENTION

The present invention relates to a temperature monitoring wearable for sleep monitoring and analysis.

It is an object of this invention to provide an artificial intelligence engine that is operable to analyze temperature data captured by a wearable.

In one embodiment, the present invention is directed to a wearable device for health-monitoring, including an armband, and at least one sensor attached to the armband, wherein the at least one sensor is operable to gather physiological data regarding an individual, wherein the at least one sensor is in network-based communication with at least one server, including a processor and a database, and wherein the processor includes an artificial intelligence module operable to determine a current health state and/or a future health state of the individual based on the physiological data.

In another embodiment, the present invention is directed to a method of health monitoring, including providing an armband including at least one sensor, the at least one sensor generating physiological data regarding an individual, the at least one sensor transmitting the physiological data to at least one server, including a processor and a database, wherein the processor includes an artificial intelligence module, and the artificial intelligence module determining a current health state and/or a future health state of the individual based on the physiological data.

In yet another embodiment, the present invention is directed to a system of health monitoring, including at least one sensor in network communication with at least one server, including a processor and a database, and an armband, wherein the at least one sensor is attached to the armband, wherein the at least one sensor is operable to generate physiological data and transmit the physiological data to the at least one server, wherein the processor includes an artificial intelligence module operable to determine a current health state and/or a future health state of an individual based on the physiological data, and wherein the processor is operable to automatically recommend a treatment plan based on the physiological data and transmit the recommended treatment plan to a user device.

These and other aspects of the present invention will become apparent to those skilled in the art after a reading of the following description of the preferred embodiment when considered with the drawings, as they support the claimed invention.

### BRIEF DESCRIPTION OF THE DRAWINGS

FIG. 1 illustrates a wearable device according to one embodiment of the present invention.

FIG. 2 illustrates is a block diagram of one embodiment of the sleep monitoring system.

FIG. 3 illustrates a welcome interface for a health monitoring application according to one embodiment of the present invention.

FIG. 4 illustrates an instruction interface for setting up a wearable device for a health monitoring system according to one embodiment of the present invention.

FIG. 5 illustrates a sync interface for an information hub according to one embodiment of the present invention.

FIG. 6 illustrates a device status interface according to one embodiment of the present invention.

FIG. 7 illustrates a temperature data interface according to one embodiment of the present invention.

FIG. 8 illustrates a temperature data interface with sleep staging information according to one embodiment of the present invention.

FIG. 9 illustrates a sleep cycle summary interface according to one embodiment of the present invention.

FIG. 10 illustrates a metrics interface according to one embodiment of the present invention.

FIG. 11 illustrates a 30-day summary chart interface according to one embodiment of the present invention.

FIG. 12 is a schematic diagram of a system of the present invention.

### DETAILED DESCRIPTION

The present invention is generally directed to a temperature monitoring wearable device with an artificial intelligence engine.

In one embodiment, the present invention is directed to a wearable device for health-monitoring, including an armband, and at least one sensor attached to the armband, wherein the at least one sensor is operable to gather physiological data regarding an individual, wherein the at least one sensor is in network-based communication with at least one server, including a processor and a database, and wherein the processor includes an artificial intelligence module operable to determine a current health state and/or a future health state of the individual based on the physiological data.

In another embodiment, the present invention is directed to a method of health monitoring, including providing an armband including at least one sensor, the at least one sensor generating physiological data regarding an individual, the at least one sensor transmitting the physiological data to at least one server, including a processor and a database, wherein the processor includes an artificial intelligence module, and the artificial intelligence module determining a current health state and/or a future health state of the individual based on the physiological data.

In yet another embodiment, the present invention is directed to a system of health monitoring, including at least one sensor in network communication with at least one server, including a processor and a database, and an armband, wherein the at least one sensor is attached to the armband, wherein the at least one sensor is operable to generate physiological data and transmit the physiological data to the at least one server, wherein the processor includes an artificial intelligence module operable to determine a current health state and/or a future health state of an individual based on the physiological data, and wherein the processor is operable to automatically recommend a treatment plan based on the physiological data and transmit the recommended treatment plan to a user device.

None of the prior art discloses a sleep monitoring system with a temperature sensing wearable device and an artificial intelligence system that is operable to analyze and model real-time biometric and physiological information.

Temperature is an important component of an individual's circadian rhythm. The circadian rhythm helps an individual transition between being awake or asleep. Typically, an individual's body temperature will drop near an optimal bedtime and rise again before waking up. Melatonin is released to help trigger the body's cooling process, but the cooling process is easily disrupted by blue light. If the average temperature is significantly above or below average or if it fails to change when attempting to fall asleep, then an individual's body is often under strain. Additionally, body temperature is related to the sleep cycle. Non-rapid eye movement (NREM) onset is typically accompanied by the steepest drop in brain temperature. Brain temperature during (rapid eye movement) REM is determined by the room temperature. Therefore, it is important to monitor an individual's temperature before, during, and after sleep to determine the quality of sleep and health of an individual.

Thermoregulatory mechanisms are fundamental to sleep and an individual's energy homeostasis. Many individuals struggle to constantly find a cool spot to sleep, which often leads to sleep deprivation. Sleep deprivation negatively affects an individual's ability to regulate their temperature circle. Sleep deprivation is also an indicator of vasospastic disorders or disorders affecting one's ability to restrict and expand veins and arteries to maintain or release heat.

Additionally, many factors affect a person's body temperature. For example, and not limitation, the type of mattress, the type of clothing and use thereof, the room temperature, the type of pillow, and the type of blanket all have an impact on the quality of sleep. However, it is difficult to correlate the impact of each factor together. Therefore, there is a need for an artificial intelligence engine that provides for modeling and analysis based off of body temperature data and other relevant sleep factors.

Referring now to the drawings in general, the illustrations are for the purpose of describing one or more preferred embodiments of the invention and are not intended to limit the invention thereto.

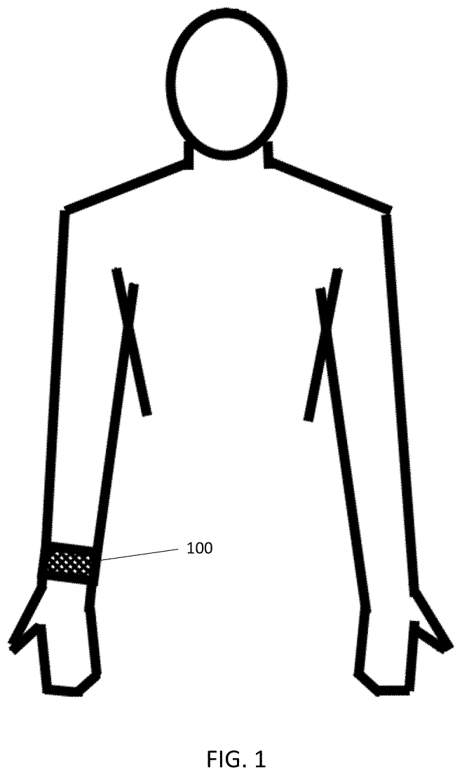

In one embodiment, the present invention includes at least one wearable device and an artificial intelligence system. According to one embodiment, as shown in FIG. 1, the wearable device is an armband 100. The armband 100 is operable to adjust in size for users of different sizes. The armband is further configured for wireless communication with at least one remote device. An armband refers to any wearable device configured to be worn around any section of a human arm, including the forearm, the upper arm, the wrist, and/or the elbow. In another embodiment, the at least one wearable device includes at least one sensor. In yet another embodiment, the at least one sensor includes a temperature sensor. The at least one wearable device is operable to capture real-time or near real-time temperature data from varying locations of a user's arm. Alternatively, for example, and not limitation, the at least one wearable device includes a hat, a shirt, a wristband, a sock, a chest band, a glove, and/or combinations thereof. The at least one wearable device is operable for continuous and discontinuous monitoring of a user. The at least one sensor is operable to collect biometric data and/or at least one physiological condition. In yet another embodiment, the biometric data and/or at least one physiological condition includes temperature data, weight data, size data, EEG data, ECG data, and environment data.

In one embodiment, the present system includes at least one information hub. The at least one wearable device includes at least one RFID chip and/or at least one other device for communicating information with the at least one information hub. In one embodiment, data generated by the at least one wearable device is automatically transmitted to the at least one information hub in real-time or near real-time when the at least one wearable device is within range of the at least one information hub. The information hub then communicates the wearable data to at least one server. In another embodiment, the at least one wearable device directly communicates data with the at least one server. In yet another embodiment, the at least one wearable device does not communicate data with the at least one information hub in real-time or near real-time, but instead communicates in defined time intervals (e.g., 30 seconds, 1 minute, 5 minutes, 10 minutes, 15 minutes, 30 minutes, 1 hour, 6 hours, 12 hours, 24 hours, etc.).

In yet another embodiment, the artificial intelligence system includes software. The software includes an artificial intelligence engine and at least one visualization tool. The artificial intelligence engine is operable to collect, correlate, integrate, synchronize, and store real-time or near real-time data. The real-time or near real-time data includes body temperature data. Advantageously, the present invention is operable to correlate the captured real-time or near real-time wearable data with at least one physiological factor, environment factor, and/or user input. The at least one physiological factor includes body weight, body fat composition, body temperature, and exercise data. The user input data includes bathing information (ex. how recently did the user shower), type of mattress, type of sheets, type of blankets, and the type and amount of clothing being worn. The environment data includes room temperature, noise, pollution and other environment data. The artificial intelligence engine is operable to determine sleep temperature patterns based on the real-time wearable data to detect the onset of fever activity. The artificial intelligence engine is further operable to use the captured real-time wearable data and at least one physiological factor, environmental factor and/or user input to provide a real-time or near real-time quality of sleep score. For example, and not limitation, the quality of sleep score is based on the number of hours a person slept, how long a person was in the REM phase of the sleep circle, how much body temperature fluctuated, how quickly a person woke up, and other factors to illustrate how well a person slept. The present invention is configured to correlate a significant change in sleep score with a corresponding change of biometric data, physiological data, and/or environment data.

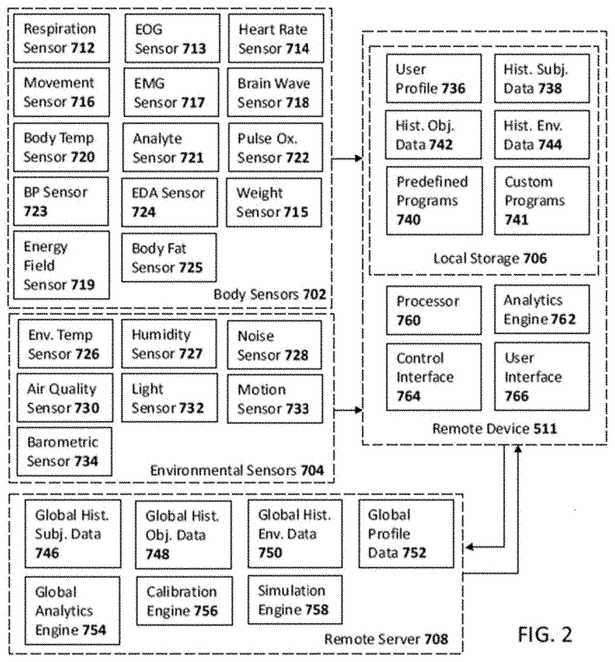

FIG. 2 is a block diagram of one embodiment of the sleep monitoring system. The sleep monitoring system 700 includes body sensors 702, environmental sensors 704, a remote device 511 with local storage 706, and a remote server 708. The body sensors 702 include a respiration sensor 712, an electrooculography (EOG) sensor 713, a heart rate sensor 714, a body weight sensor 715, a movement sensor 716, an electromyography (EMG) sensor 717, a brain wave sensor 718, an energy field sensor 719, a body temperature sensor 720, an analyte sensor 721, a pulse oximeter sensor 722, a blood pressure (BP) sensor 723, an electrodermal activity (EDA) sensor 724, and/or a body fat sensor 725.

The electrooculography (EOG) sensor 713 measures the corneo-retinal standing potential that exists between the front and the back of the eye. Measurements of eye movements are done by placing pairs of electrodes either above and below the eye or to the left and right of the eye. If the eye moves to a position away from the center and toward one of the electrodes, a potential difference occurs between the electrodes. The recorded potential is a measure of the eye's position. In one embodiment, the present invention is operable to compare the eye's position and movement data in real-time to determine a user's position in the sleep cycle. Advantageously, the present invention is operable to correlate temperature data with the eye position and movement data in real-time to determine how temperature is affecting a user's sleep.

The heart rate sensor 714 is preferably incorporated into the at least one wearable. Alternatively, the heart rate sensor 714 is attached to the user with a chest strap. In another embodiment, the heart rate sensor 714 is incorporated into a patch or a bandage. The heart rate is determined using electrocardiography, pulse oximetry, ballistocardiography, or seismocardiography. In one embodiment, the heart rate sensor 714 measures heart rate variability (HRV). HRV is a measurement of the variation in time intervals between heartbeats. A high HRV measurement is indicative of less stress, while a low HRV measurement is indicative of more stress. Studies have linked abnormalities in HRV to diseases where stress is a factor (e.g., diabetes, depression, congestive heart failure). In one embodiment, a Poincaré plot is generated to display HRV on a device such as a smartphone.

The artificial intelligence system of the present invention is operable to correlate the heart rate data with other biometric data. For example, and not limitation, the artificial intelligence engine is operable to correlate the heart rate data with changes in the body temperature, environment data and movement data.

The body weight sensor 715 is preferably a smart scale (e.g., Fitbit® Aria®, Nokia® Body+, Garmin® Index™, Under Armour® Scale, Pivotal Living® Smart Scale, iHealth® Core). Alternatively, the body weight sensor 715 is at least one pressure sensor embedded in a mat, a mattress or a mattress topper. In one embodiment, the sleep monitoring system 700 is also operable to determine a height of a user using the at least one pressure sensor embedded in a mat, a mattress or a mattress topper. In another embodiment, a body mass index (BMI) of the user is calculated using the body weight of the user and the height of the user as measured by the at least one pressure sensor.

The movement sensor 716 is an accelerometer and/or a gyroscope. In one embodiment, the accelerometer and/or the gyroscope are incorporated into at least one wearable device. In another embodiment, the accelerometer and/or the gyroscope are incorporated into a smartphone. In alternative embodiment, the movement sensor 716 is a non-contact sensor. In one embodiment, the movement sensor 716 is at least one piezoelectric sensor. In another embodiment, the movement sensor 716 is a pyroelectric infrared sensor (i.e., a “passive” infrared sensor). In yet another embodiment, the movement sensor 716 is at least one pressure sensor embedded in a mattress or mattress topper. Alternatively, the movement sensor 716 is incorporated into a smart fabric.

The electromyography (EMG) sensor 717 records the electrical activity produced by skeletal muscles. Impulses are recorded by attaching electrodes to the skin surface over the muscle. In a preferred embodiment, three electrodes are placed on the chin. One in the front and center and the other two underneath and on the jawbone. These electrodes demonstrate muscle movement during sleep, which are able to be used to detect REM or NREM sleep. In another embodiment, two electrodes are placed on the inside of each calf muscle about 2 to 4 cm (about 0.8 to 1.6 inches) apart. In yet another embodiment, two electrodes are placed over the anterior tibialis of each leg. The electrodes on the leg are used to detect movement of the legs during sleep, which sometimes occurs with Restless Leg Syndrome or Periodic Limb Movements of Sleep.

The brain wave sensor 718 is preferably an electroencephalogram (EEG) with at least one channel. In a preferred embodiment, the EEG has at least two channels. Multiple channels provide higher resolution data. The frequencies in EEG data indicate particular brain states. The brain wave sensor 718 is preferably operable to detect delta, theta, alpha, beta, and gamma frequencies. In another embodiment, the brain wave sensor 718 is operable to identify cognitive and emotion metrics, including focus, stress, excitement, relaxation, interest, and/or engagement. In yet another embodiment, the brain wave sensor 718 is operable to identify cognitive states that reflect the overall level of engagement, attention and focus and/or workload that reflects cognitive processes (e.g., working memory, problem solving, analytical reasoning).

The energy field sensor 719 measures an energy field of a user. In one embodiment, the energy field sensor 719 is a gas discharge visualization (GDV) device. Examples of a GDV device are disclosed in U.S. Pat. Nos. 7,869,636 and 8,321,010 and U.S. Publication No. 20100106424, each of which is incorporated herein by reference in its entirety. The GDV device utilizes the Kirlian effect to evaluate an energy field. In a preferred embodiment, the GDV device utilizes a high-intensity electric field (e.g., 1024 Hz, 10 kV, square pulses) input to an object (e.g., human fingertips) on an electrified glass plate. The high-intensity electric field produces a visible gas discharge glow around the object (e.g., fingertip). The visible gas discharge glow is detected by a charge-coupled detector and analyzed by software on a computer. The software characterizes the pattern of light emitted (e.g., brightness, total area, fractality, density). In a preferred embodiment, the software utilizes Mandel's Energy Emission Analysis and the Su-Jok system of acupuncture to create images and representations of body systems. The energy field sensor 719 is preferably operable to measure stress levels, energy levels, and/or a balance between the left and right sides of the body.

The body temperature sensor 720 measures core body temperature and/or skin temperature in real-time. The body temperature sensor 720 is a thermistor, an infrared sensor, or thermal flux sensor. In one embodiment, the body temperature sensor 720 is incorporated into an armband or a wristband. In another embodiment, the body temperature sensor 720 is incorporated into a patch or a bandage. In yet another embodiment, the body temperature sensor 720 is an ingestible core body temperature sensor (e.g., CorTemp®). The body temperature sensor 720 is preferably wireless.

The analyte sensor 721 monitors levels of an analyte in blood, sweat, or interstitial fluid. In one embodiment, the analyte is an electrolyte, a small molecule (molecular weight <900 Daltons), a protein (e.g., C-reactive protein), and/or a metabolite. In another embodiment, the analyte is glucose, lactate, glutamate, oxygen, sodium, chloride, potassium, calcium, ammonium, copper, magnesium, iron, zinc, creatinine, uric acid, oxalic acid, urea, ethanol, an amino acid, a hormone (e.g., cortisol, melatonin), a steroid, a neurotransmitter, a catecholamine, a cytokine, and/or an interleukin (e.g., IL-6). The analyte sensor 721 is preferably non-invasive. Alternatively, the analyte sensor 721 is minimally invasive or implanted. In one embodiment, the analyte sensor 721 is incorporated into a wearable device. Alternatively, the analyte sensor 721 is incorporated into a patch or a bandage.

The pulse oximeter sensor 722 monitors oxygen saturation. In one embodiment, the pulse oximeter sensor 722 is worn on a finger, a toe, or an ear. In another embodiment, the pulse oximeter sensor 722 is incorporated into a patch or a bandage. The pulse oximeter sensor 722 is preferably wireless. Alternatively, the pulse oximeter sensor 722 is wired. In one embodiment, the pulse oximeter sensor 722 is connected by a wire to a wrist strap or a strap around a hand. In another embodiment, the pulse oximeter sensor 722 is combined with a heart rate sensor 714. In yet another embodiment, the pulse oximeter sensor 722 uses a camera lens on a smartphone or a tablet.

The blood pressure (BP) sensor 723 is a sphygmomanometer. The sphygmomanometer is preferably wireless. Alternatively, the blood pressure sensor 723 estimates the blood pressure without an inflatable cuff (e.g., Salu™ Pulse+). In one embodiment, the blood pressure sensor 723 is incorporated into a wearable device.

The electrodermal activity sensor 724 measures sympathetic nervous system activity. Electrodermal activity is more likely to have high frequency peak patterns (i.e., “storms”) during deep sleep. In one embodiment, the electrodermal activity sensor 724 is incorporated into a wearable device. Alternatively, the electrodermal activity sensor 724 is incorporated into a patch or a bandage.

The body fat sensor 725 is preferably a bioelectrical impedance device. In one embodiment, the body fat sensor 725 is incorporated into a smart scale (e.g., Fitbit® Aria®, Nokia® Body+, Garmin® Index™, Under Armour® Scale, Pivotal Living® Smart Scale, iHealth® Core). Alternatively, the body fat sensor 725 is a handheld device.

The environmental sensors 704 include an environmental temperature sensor 726, a humidity sensor 727, a noise sensor 728, an air quality sensor 730, a light sensor 732, a motion sensor 733, and/or a barometric sensor 734. In one embodiment, the environmental temperature sensor 726, the humidity sensor 727, the noise sensor 728, the air quality sensor 730, the light sensor 732, the motion sensor 733, and/or the barometric sensor 734 are incorporated into a home automation system (e.g., Amazon® Alexa®, Apple® HomeKit™, Google® Home™, IF This Then That® (IFTTT®), Nest®). Alternatively, the environmental temperature sensor 726, the humidity sensor 727, the noise sensor 728, and/or the light sensor 732 are incorporated into a smartphone or tablet. In one embodiment, the noise sensor 728 is a microphone. In one embodiment, the air quality sensor 730 measures carbon monoxide, carbon dioxide, nitrogen dioxide, sulfur dioxide, particulates, and/or volatile organic compounds (VOCs).

The remote device 511 is preferably a smartphone or a tablet. Alternatively, the remote device 511 is a laptop or a desktop computer. The remote device 511 includes a processor 760, an analytics engine 762, a control interface 764, and a user interface 766. The remote device 511 accepts data input from the body sensors 702 and/or the environmental sensors 704. The remote device also accepts data input from the remote server 708. The remote device 511 stores data in a local storage 706.

The local storage 706 on the remote device 511 includes a user profile 736, historical subjective data 738, predefined programs 740, custom programs 741, historical objective data 742, and historical environmental data 744. The user profile 736 stores sleep monitoring system preferences and information about the user, including but not limited to, age, weight, height, gender, medical history (e.g., sleep conditions, medications, diseases), fitness (e.g., fitness level, fitness activities), sleep goals, stress level, and/or occupational information (e.g., occupation, shift information). The medical history includes caffeine consumption, alcohol consumption, tobacco consumption, use of prescription sleep aids and/or other medications, blood pressure, restless leg syndrome, narcolepsy, headaches, heart disease, sleep apnea, depression, stroke, diabetes, insomnia, anxiety or post-traumatic stress disorder (PTSD), and/or neurological disorders.

In one embodiment, the medical history incorporates information gathered from the Epworth Sleepiness Scale (ESS), the Insomnia Severity Index (ISI), Generalized Anxiety Disorder 7-item (GAD-7) Scale, and/or Patient Heath Questionanaire-9 (PHQ-9) (assessment of depression). The ESS is described in Johns MW (1991). “A new method for measuring da sleepiness: the Epworth sleepiness scale”, Sleep, 14 (6): 540-5, is incorporated herein by reference in its entirety. The ISI is described in Morin et al. (2011). “The Insomnia Severity Index: Psychometric Indicators to Detect Insomnia Cases and Evaluate Treatment Response”, Sleep, 34(5): 601-608, which is incorporated herein by reference in its entirety. The GAD 7 is described in Spitzer et al., “A brief measure for assessing generalized anxiety disorder: the GAD-7”, Arch Intern Med., 2006 May 22; 166(1):1092-7, which is incorporated herein by reference in its entirety. The PHQ-9 is described in Kroenke et al., “The PHQ-9: Validity of a Brief Depression Severity Measure”, J. Gen. Intern. Med., 2001 September; 16(9): 606-613, which is incorporated herein by reference in its entirety.

In one embodiment, the weight of the user is automatically uploaded to the local storage from a third-party application. In one embodiment, the third-party application obtains the information from a smart scale (e.g., Fitbit® Aria®, Nokia® Body+™ Garmin® Index™ Under Armour® Scale, Pivotal Living® Smart Scale, iHealth® Core). In another embodiment, the medical history includes information gathered from a Resting Breath Hold test.

The historical objective data 742 includes information gathered from the body sensors 702. This includes information from the respiration sensor 712, the electrooculography sensor 713, the heart rate sensor 714, the movement sensor 716, the electromyography sensor 717, the brain wave sensor 718, the energy field sensor 719, the body temperature sensor 720, the analyte sensor 721, the pulse oximeter sensor 722, the blood pressure sensor 723, and/or the electrodermal activity sensor 724. In another embodiment, the historical objective data 742 includes information gathered from the Maintenance of Wakefulness Test, the Digit Symbol Substitution Test, and/or the Psychomotor Vigilance Test. The Maintenance of Wakefulness Test is described in Doghramji, et al., “A normative study of the maintenance of wakefulness test (MWT)”, Electroencephalogr. Clin. Neurophysiol., 1997 November; 103(5): 554-562, which is incorporated herein by reference in its entirety. The Digit Symbol Substitution Test is described in Wechsler, D. (1997). Wechsler Adult Intelligence Scale—Third edition (WAIS-III). San Antonio, Tex.: Psychological Corporation and Wechsler, D. (1997). Wechsler Memory Scale—Third edition (WMS-III). San Antonio, Tex.: Psychological Corporation, each of which is incorporated herein by reference in its entirety. The Psychomotor Vigilance Test is described in Basner et al., “Maximizing sensitivity of the psychomotor vigilance test (PVT) to sleep loss”, Sleep, 2011 May 1; 34(5): 581-91, which is incorporated herein by reference in its entirety.

The historical environmental data 744 includes information gathered from the environmental sensors 704. This includes information from the environmental temperature sensor 726, the humidity sensor 727, the noise sensor 728, the air quality sensor 730, the light sensor 732, and/or the barometric sensor 734.

The historical subjective data 738 includes information regarding sleep and/or stress. In one embodiment, the information regarding sleep is gathered from manual sleep logs (e.g., Pittsburgh Sleep Quality Index). The manual sleep logs include, but are not limited to, a time sleep is first attempted, a time to fall asleep, a time of waking up, hours of sleep, number of awakenings, times of awakenings, length of awakenings, perceived sleep quality, use of medications to assist with sleep, difficulty staying awake and/or concentrating during the day, difficulty with temperature regulation at night (e.g., too hot, too cold), trouble breathing at night (e.g., coughing, snoring), having bad dreams, waking up in the middle of the night or before a desired wake up time, twitching or jerking in the legs while asleep, restlessness while asleep, difficulty sleeping due to pain, and/or needing to use the bathroom in the middle of the night. The Pittsburgh Sleep Quality Index is described in Buysse, et al., “The Pittsburgh sleep quality index: A new instrument for psychiatric practice and research”, Psychiatry Research. 28 (2): 193-213 (May 1989), which is incorporated herein by reference in its entirety.

In another embodiment, the historical subjective data 738 includes information gathered regarding sleepiness (e.g., Karolinska Sleepiness Scale, Stanford Sleepiness Scale, Epworth Sleepiness Scale). The Karolinska Sleepiness Scale is described in Åkerstedt, et al., “Subjective and objective sleepiness in the active individual”, Int J Neurosc., 1990; 52:29-37 and Baulk et al., “Driver sleepiness—evaluation of reaction time measurement as a secondary task”, Sleep, 2001; 24(6):695-698, each of which is incorporated herein by reference in its entirety. The Stanford Sleepiness Scale is described in Hoddes E. (1972). “The development d use of the stanford sleepiness scale (SSS)”. Psychophysiology. 9 (150) and Maclean, et al, (1992-03-01). “Psychometric evaluation of the Stanford Sleepiness Scale”. Journal of Sleep Research. 1 (1): 35-39, each of which is incorporated herein by reference in its entirety.

In yet another embodiment, the historical subjective data 738 includes information regarding tension or anxiety, depression or dejection, anger or hostility, and/or fatigue or inertia gathered from the Profile of Mood States. The Profile of Mood States is described in the Profile of Mood States, 2^{nd} Edition published by Multi-Health Systems (2012) and Curran et al., “Short Form of the Profile of Mood States (POMS-SF): Psychometric information”, Psychological Assessment. 7 (1): 80-83 (1995), each of which is incorporated herein by reference in its entirety. In another embodiment, the historical subjective data 738 includes information gathered from the Ford Insomnia Response to Stress Test (FIRST), which asks how likely a respondent is to have difficulty sleeping in nine different situations. The FIRST is described in Drake et al., “Vulnerability to stress-related sleep disturbance and hyperarousal”, Sleep, 2004; 27:285-91 and Drake et al., “Stress-related sleep disturbance and polysomnographic response to caffeine”, Sleep Med., 2006; 7:567-72, each of which is incorporated herein by reference in its entirety. In still another embodiment, the historical subjective data 738 includes information gathered from the Impact of Events, which assesses the psychological impact of stressful life events. A subscale score is calculated for intrusion, avoidance, and/or hyperarousal. The Impact of Events is described in Weiss, D. S., & Marmar, C. R. (1996). The Impact of Event Scale-Revised. In J. Wilson & T. M. Keane (Eds.), Assessing psychological trauma and PTSD (pp. 399-411). New York: Guilford, which is incorporated herein by reference in its entirety. In one embodiment, the historical subjective data 738 includes information gathered from the Social Readjustment Rating Scale (SRRS). The SRRS lists 52 stressful life events and assigns a point value based on how traumatic the event was determined to be by a sample population. The SRRS is described in Holmes et al., “The Social Readjustment Rating Scale”, J. Psychosom. Res. 11(2): 213-8 (1967), which is incorporated herein by reference in its entirety.

The remote server 708 includes global historical subjective data 746, global historical objective data 748, global historical environmental data 750, global profile data 752, a global analytics engine 754, a calibration engine 756, and a simulation engine 758. The global historical subjective data 746, the global historical objective data 748, the global historical environmental data 750, and the global profile data 752 include data from multiple users.

The body sensors 702, the environmental sensors 704, the remote device 511 with local storage 706, and the remote server 708 are designed to connect directly (e.g., Universal Serial Bus (USB) or equivalent) or wirelessly (e.g., Bluetooth®, Wi-Fi®, ZigBee®) through systems designed to exchange data between various data collection sources. In a preferred embodiment, the body sensors 702, the environmental sensors 704, the remote device 511 with local storage 706, and the remote server 708 communicate wirelessly through Bluetooth®. Advantageously, Bluetooth® emits lower electromagnetic fields (EMFs) than Wi-Fi® and cellular signals.

The artificial intelligence engine is operable to receive real-time or near real-time data from the body and environment sensors. In one embodiment, at least one remote device includes the artificial intelligence engine. The at least one remote device includes a cellphone, a laptop, a tablet, a desktop computer and other remote devices. The artificial intelligence engine includes a visualization component. The visualization component is operable to display the captured and analyzed real-time or near real-time data. The visualization tool is operable to generate and display bar graphs, line graphs, circle graphs, histograms, mosaic charts, a spider chart, a flow chart, a control chart, a waterfall chart, a scatter plot, anatomical diagrams, and other graphical displays to illustrate the captured data.

The artificial intelligence engine is operable to generate a baseline health score based on the received biometric and physiological data. The at least one remote device is operable to receive user input for biometric and physiological user information. The artificial intelligence engine is operable to generate a user's real-time or near real-time baseline temperature before, during, and after sleep. This allows for a user to determine how much a user's temperature changes during sleep. Additionally, the artificial intelligence engine is operable to correlate the real-time or near real-time data with different stages of the sleep cycle. Generally during Phase 1 and 2 of the sleep cycle, heart rate and breathing slows down and body temperature begins to drop. During Phase 3 and 4, blood pressure drops and long, slow brain waves occur. Using the baseline data, the present invention is operable to determine when a user enters into each phase as well as how much a user's biometric and physiological data changes during each phase. This improves the real-time or near real-time analysis and allows for the present invention to generate real-time or near real-time alerts if a user is experiencing sleep conditions during unsafe situations (ex. driving). For example, and not limitation, a user's temperature will began to drop around 2 p.m. in the afternoon to prepare for sleep. The present invention is operable to recognize the preparation for sleep and to model and determine when the user is actually going to sleep during the night by comparing the real-time data. The artificial intelligence engine is operable to use historical data to provide prompts and/or questions to a user to improve analysis. Additionally, the artificial intelligence engine is operable to provide user specific questions based on family data and changes in a user's biometric and physiological data. Advantageously, the present invention is operable to compare a user's symptoms to previous medical data for other patients that suffered from similar symptoms, diseases or other shared characteristics such as age, weight, height, and/or gender.

In yet another embodiment, the artificial intelligence system is operable to generate a real-time or near real-time alert based on the real-time or near real-time biometric and physiological data, environment data, and/or user input data. The at least one alert includes minimal temperature variance during sleep, a massive drop of temperature during sleep, high brain activity during sleep, changes in heart rate, changes in environmental conditions (ex. room temperature), sleeping in a poor position and other similar alerts relating to a user's sleep. Additionally, the present invention is configured to provide a recommendation based on the alert. By way of example and not limitation, if the artificial intelligence system determines that the only significant change with a user's data was an increased body temperature and the only other change was an increase in room temperature during the night, then the present invention is operable to recommend increasing fan speed or opening a window or other methods of decreasing the temperature in the room. The present invention is further operable to recommend a treatment plan and/or a physician based on user data. For example, and not limitation, the present invention is operable to detect the onset of a fever and provide an alert of early fever detection as a warning sign of infection. Advantageously, this allows for a user to receive real-time feedback on their health condition and to take proactive steps to prevent and/or address potentially harmful conditions. Additionally, the present invention is operable to use the change in temperature data to detect early signs of diseases (ex. COVID-19). The present invention is operable to generate an alert to get tested for a particular disease and provide a clinic and/or medical facility that is able to provide the treatment.

In one embodiment, a plurality of wristbands and/or armbands, each associated with at least one sensor, are associated with a single administrator profile. A graphical user interface (GUI) is generated for user devices associated with the administrator profile, allowing an administrator to view data and analytics pertaining to each of the plurality of wristbands and/or armbands. By allowing an administrator to keep track of the health data of each user, the system better allows for management of user health. By way of example and not of limitation, in one embodiment, the administrator profile is controlled by a coach of at least one player, a parent of at least one child, and/or a physician of at least one patient.

For further detail regarding the artificial intelligence engine, see U.S. Pat. No. 10,086,231 & U.S. Pat. No. 10,471,304, each of which is incorporated herein by reference in its entirety.

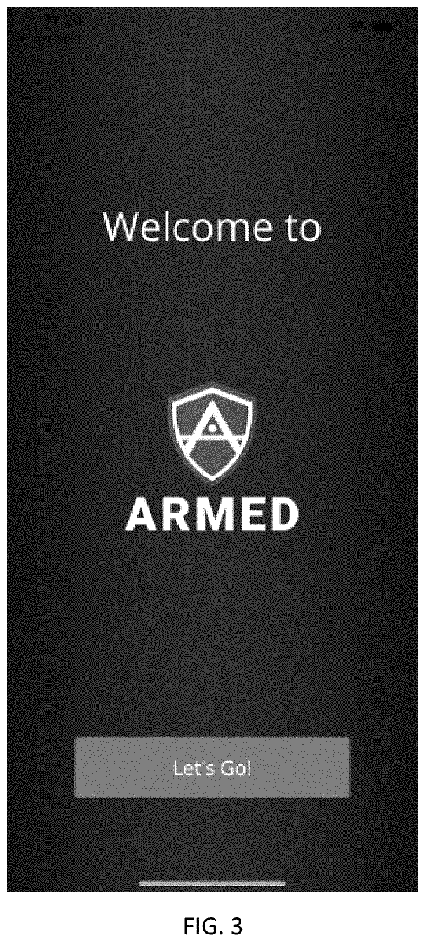

FIG. 3 illustrates a welcome interface for a health monitoring application according to one embodiment of the present invention. In one embodiment, the wristband and/or armband is operable to provide data to a server platform. The server platform generates a graphic user interface (GUI) operable to display on a user device (e.g., a phone, a tablet, a computer, a smart watch, etc.). In one embodiment, upon opening the application, a welcome interface is generated including at least one object operable to receive an input from the user device to advance beyond the welcome screen. In one embodiment, the welcome interface is only generated when the application is first opened on a user device. In another embodiment, the welcome interface is generated time an application is opened on a user device.

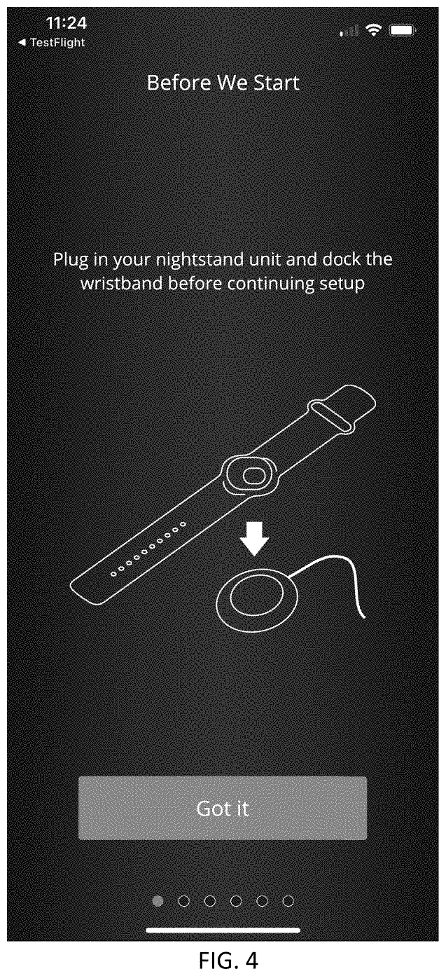

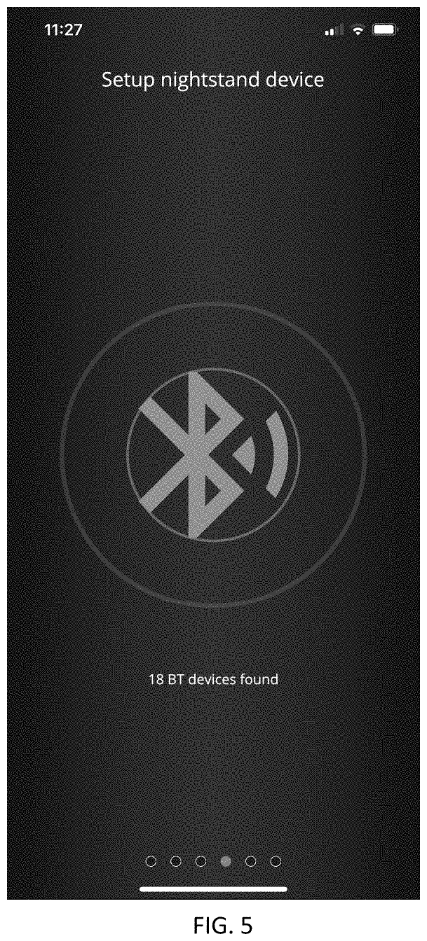

FIG. 4 illustrates an instruction interface for setting up a wearable device for a health monitoring system according to one embodiment of the present invention. In one embodiment, the application provides an instruction interface for setting up a wearable device (e.g., an armband and/or a wristband) to communicate information to the server platform. In one embodiment, set-up for the present invention includes bringing the wearable device in close proximity to an information hub while both devices are in sync mode. In one embodiment, each of the wearable device and the information hub include at least one button operable to activate sync mode. Activating the button for each the wearable device and the information hub at approximating the same time when both devices are in close proximity allows the devices to sync. In another embodiment, the wearable device and/or the information hub are constantly able to accept and/or transmit signals (e.g., BLUETOOTH signals). As shown in FIG. 5, in one embodiment, the application includes a sync interface that allows the user device to find and detect the wearable device and the information hub. In one embodiment, the sync interface is operable to accept an input from a user device selecting a nearby wearable device and a nearby information hub. Based on the selections made through the sync interface, the wearable device and the information hub are synced.

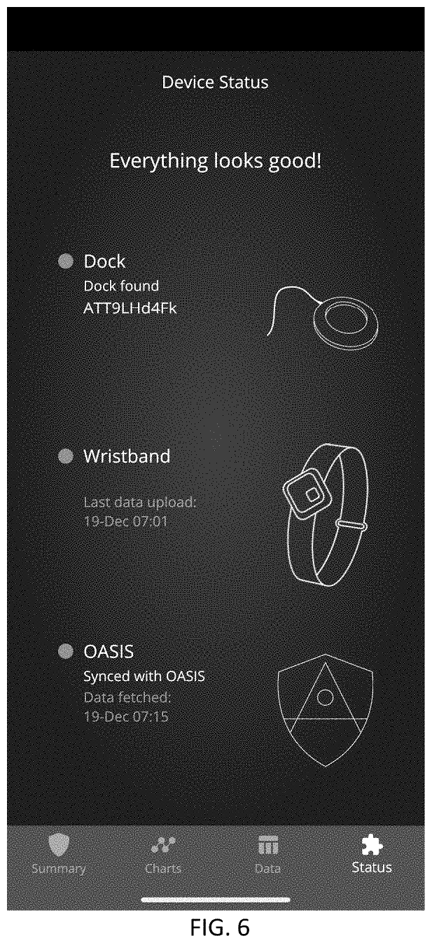

FIG. 6 illustrates a device status interface according to one embodiment of the present invention. The device status interface provides a list of devices associated with a user profile, wherein the user profile is associated with the user device on which the device status interface is presented. In one embodiment, the device status interface provides the last recorded instance in which an information hub, a wearable device, and/or a server successfully transmitted or received data. In one embodiment, if a device is unable to connect to the server platform, then the device status interface will present an error code for the device. In one embodiment, the device status interface includes a ping functionality. When the device status interface receives a selection to initiate the ping functionality, the user device will attempt to transmit a signal to a wearable device, an information hub, and/or a server and detect whether the device successfully receives the signal and transmits a signal back to the user device in response. If a signal is successfully transmitted back to the user device, then the device status interface will indicate a successful test has been performed for the particular device. If a signal is not successfully transmitted back to the user device (or not transmitted back within a predetermined time period), then the device status interface will indicate an error with regard to that device.

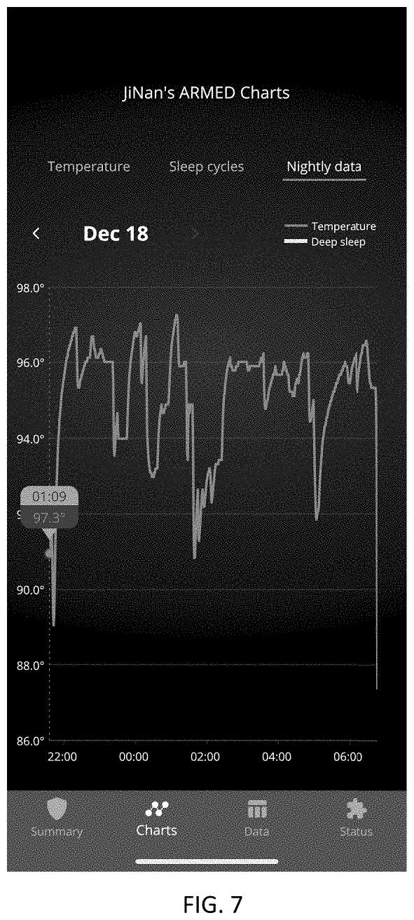

FIG. 7 illustrates a temperature data interface according to one embodiment of the present invention. In one embodiment, the application provides a temperature data interface, displaying the core body temperature of a user over time. In one embodiment, the temperature data interface is operable to receive a selection of a particular time point on a graph of the core body temperature over time. In one embodiment, the temperature data interface is operable to provide a time point and a core body temperature corresponding to the selection of the particular time point. In one embodiment, the temperature data interface is operable to receive a selection of a time range for the graph of the core body temperature over time. For example, in one embodiment, the temperature data interface is able to display core body temperature over the course of 30 minutes, an hour, 2 hours, 4 hours, 8 hours, 12 hours, 24 hours, one week, one month, one year, or any more particular time range upon selection of the time range.

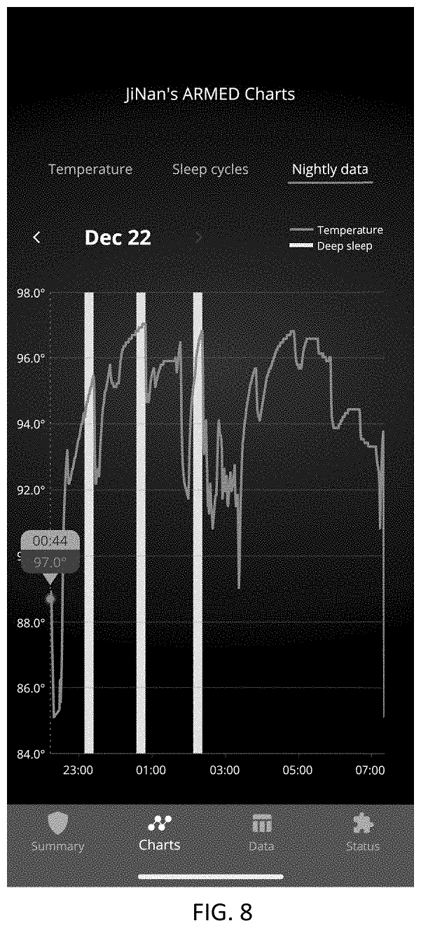

In one embodiment, as shown in FIG. 8, the graph of the core body temperature over time is also able to provide information regarding sleep stages of a user. By way of example and not of limitation, a graph of core body temperature over time is able to indicate time during deep sleep, a point of entering deep sleep, and/or a point of exiting deep sleep using vertical bars on the graph.

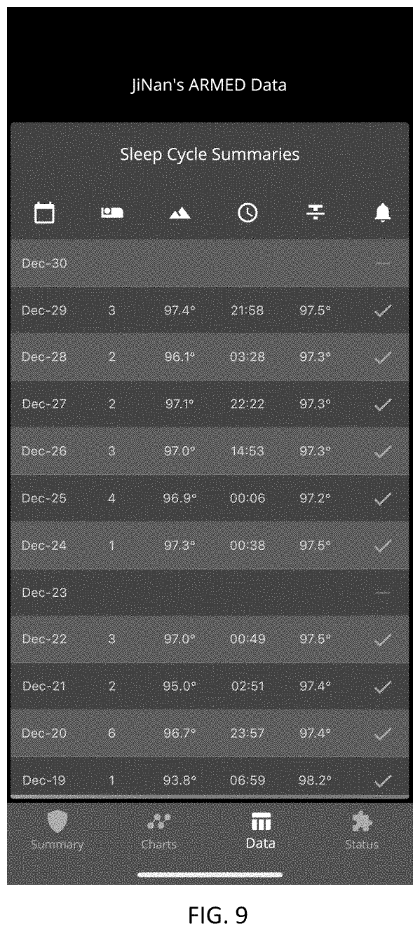

FIG. 9 illustrates a sleep cycle summary interface according to one embodiment of the present invention. In one embodiment, the application includes a sleep cycle summary interface, which provides information regarding sleep parameters for different time periods. By way of example and not of limitation, the sleep cycle summary interface is operable to provide an average core body temperature, a peak core body temperature, an alarm temperature, an average alarm temperature, an amount of time sleeping, an amount of time in deep sleep, a time first sleeping, a time first entering deep sleep, and/or other information regarding sleep cycles for a particular time period. Additionally, the sleep cycle summary interface is operable to show whether the user utilized the wearable device during each time period. In one embodiment, the different time periods are different days. In another embodiment, the different time periods are hours, days, weekends, weeks, months, years, and/or other time periods.

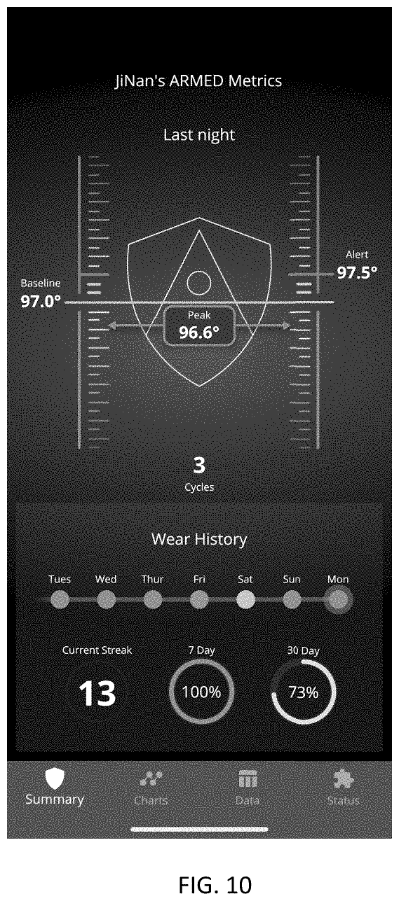

FIG. 10 illustrates a metrics interface according to one embodiment of the present invention. In one embodiment, the application provides a metrics interface, providing information regarding core body temperature during a particular time period, number of days consecutively utilizing the wearable device, and/or other information. In one embodiment, the metrics interface displays the number of consecutive days wearing the wearable device, a percentage of the past week during which the wearable device was worn, and/or a percentage of the past 30 days during which the wearable device was worn. In one embodiment, the metrics interface includes a timeline displaying a number of past days, with color coding and/or other forms of coding indicating whether the wearable device was used during each of the past days. In one embodiment, the metrics interface is operable to receive a selection of one or more of the past days. Based on the selection of the one or more of the past days, the application presents a graph of the core body temperature during a time period during the one or more of the past days.

In one embodiment, the metrics interface provides a baseline core body temperature for a user, a peak core body temperature, and/or an alarm core body temperature for a user during a particular past time period (e.g., the previous night). In one embodiment, the metrics interface is operable to receive a selection of a new alarm temperature for a subsequent time period.

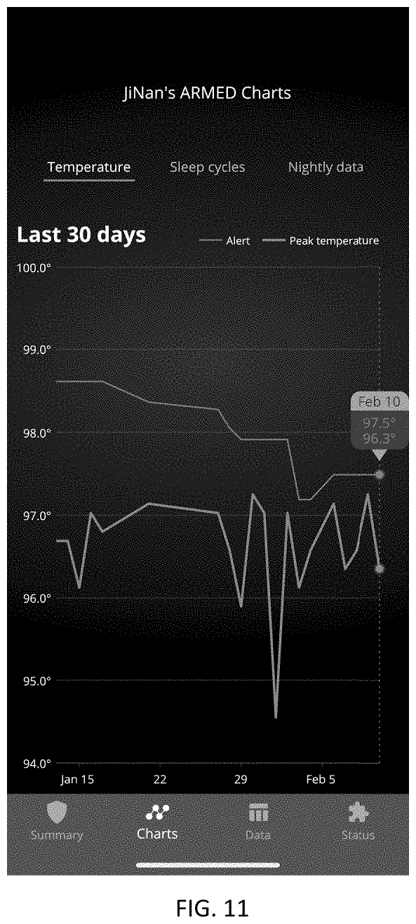

FIG. 11 illustrates a 30-day summary chart interface according to one embodiment of the present invention. In one embodiment, the application provides a 30-day summary chart interface displaying peak (or maximum) core body temperature and an alarm temperature for each time period across a 30-day time period. An alarm temperature is a temperature that indicates the possible onset of a fever. In one embodiment, an alarm temperature is a preset core body temperature by a user for a particular time period. In another embodiment, the artificial intelligence system automatically determines an alarm temperature for a user for a particular time period based on the calculated baseline temperature for the user. In one embodiment, when the core body temperature of a user exceeds the alarm temperature during a particular time period, an alert is sent to the user device of the user. In another embodiment, an alert is sent to an administrator device associated with the user.

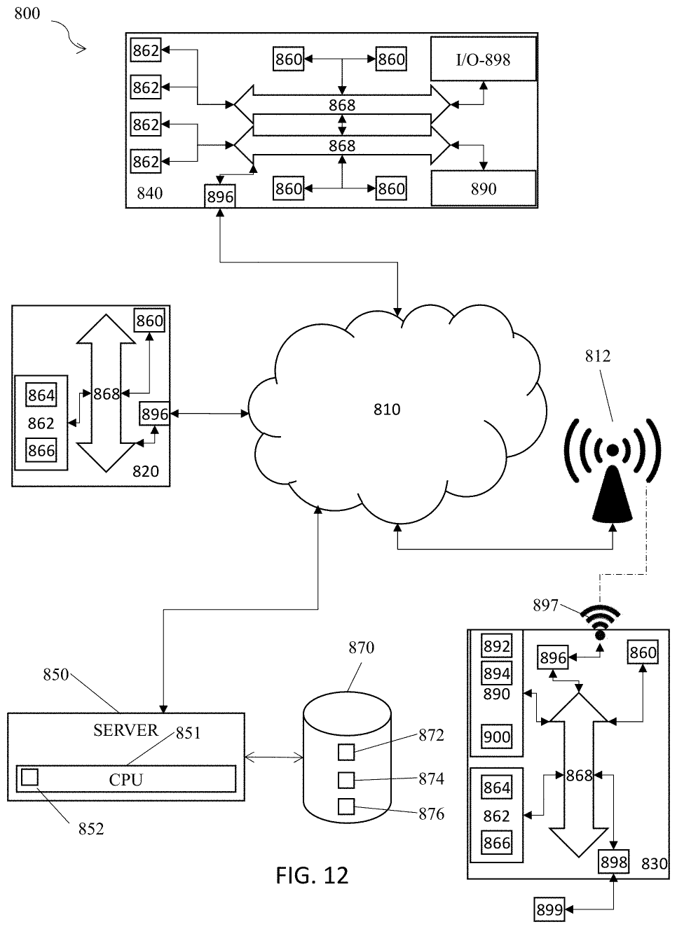

FIG. 12 is a schematic diagram of an embodiment of the invention illustrating a computer system, generally described as 800, having a network 810, a plurality of computing devices 820, 830, 840, a server 850, and a database 870.

The server 850 is constructed, configured, and coupled to enable communication over a network 810 with a plurality of computing devices 820, 830, 840. The server 850 includes a processing unit 851 with an operating system 852. The operating system 852 enables the server 850 to communicate through network 810 with the remote, distributed user devices. Database 870 is operable to house an operating system 872, memory 874, and programs 876.

In one embodiment of the invention, the system 800 includes a network 810 for distributed communication via a wireless communication antenna 812 and processing by at least one mobile communication computing device 830. Alternatively, wireless and wired communication and connectivity between devices and components described herein include wireless network communication such as WI-FI, WORLDWIDE INTEROPERABILITY FOR MICROWAVE ACCESS (WIMAX), Radio Frequency (RF) communication including RF identification (RFID), NEAR FIELD COMMUNICATION (NFC), BLUETOOTH including BLUETOOTH LOW ENERGY (BLE), ZIGBEE, Infrared (IR) communication, cellular communication, satellite communication, Universal Serial Bus (USB), Ethernet communications, communication via fiber-optic cables, coaxial cables, twisted pair cables, and/or any other type of wireless or wired communication. In another embodiment of the invention, the system 800 is a virtualized computing system capable of executing any or all aspects of software and/or application components presented herein on the computing devices 820, 830, 840. In certain aspects, the computer system 800 is operable to be implemented using hardware or a combination of software and hardware, either in a dedicated computing device, or integrated into another entity, or distributed across multiple entities or computing devices.

By way of example, and not limitation, the computing devices 820, 830, 840 are intended to represent various forms of electronic devices including at least a processor and a memory, such as a server, blade server, mainframe, mobile phone, personal digital assistant (PDA), smartphone, desktop computer, netbook computer, tablet computer, workstation, laptop, and other similar computing devices. The components shown here, their connections and relationships, and their functions, are meant to be exemplary only, and are not meant to limit implementations of the invention described and/or claimed in the present application.

In one embodiment, the computing device 820 includes components such as a processor 860, a system memory 862 having a random access memory (RAM) 864 and a read-only memory (ROM) 866, and a system bus 868 that couples the memory 862 to the processor 860. In another embodiment, the computing device 830 is operable to additionally include components such as a storage device 890 for storing the operating system 892 and one or more application programs 894, a network interface unit 896, and/or an input/output controller 898. Each of the components is operable to be coupled to each other through at least one bus 868. The input/output controller 898 is operable to receive and process input from, or provide output to, a number of other devices 899, including, but not limited to, alphanumeric input devices, mice, electronic styluses, display units, touch screens, signal generation devices (e.g., speakers), or printers.

By way of example, and not limitation, the processor 860 is operable to be a general-purpose microprocessor (e.g., a central processing unit (CPU)), a graphics processing unit (GPU), a microcontroller, a Digital Signal Processor (DSP), an Application Specific Integrated Circuit (ASIC), a Field Programmable Gate Array (FPGA), a Programmable Logic Device (PLD), a controller, a state machine, gated or transistor logic, discrete hardware components, or any other suitable entity or combinations thereof that can perform calculations, process instructions for execution, and/or other manipulations of information.

In another implementation, shown as 840 in FIG. 12, multiple processors 860 and/or multiple buses 868 are operable to be used, as appropriate, along with multiple memories 862 of multiple types (e.g., a combination of a DSP and a microprocessor, a plurality of microprocessors, one or more microprocessors in conjunction with a DSP core).

Also, multiple computing devices are operable to be connected, with each device providing portions of the necessary operations (e.g., a server bank, a group of blade servers, or a multi-processor system). Alternatively, some steps or methods are operable to be performed by circuitry that is specific to a given function.

According to various embodiments, the computer system 800 is operable to operate in a networked environment using logical connections to local and/or remote computing devices 820, 830, 840 through a network 810. A computing device 830 is operable to connect to a network 810 through a network interface unit 896 connected to a bus 868. Computing devices are operable to communicate communication media through wired networks, direct-wired connections or wirelessly, such as acoustic, RF, or infrared, through an antenna 897 in communication with the network antenna 812 and the network interface unit 896, which are operable to include digital signal processing circuitry when necessary. The network interface unit 896 is operable to provide for communications under various modes or protocols.

In one or more exemplary aspects, the instructions are operable to be implemented in hardware, software, firmware, or any combinations thereof. A computer readable medium is operable to provide volatile or non-volatile storage for one or more sets of instructions, such as operating systems, data structures, program modules, applications, or other data embodying any one or more of the methodologies or functions described herein. The computer readable medium is operable to include the memory 862, the processor 860, and/or the storage media 890 and is operable be a single medium or multiple media (e.g., a centralized or distributed computer system) that store the one or more sets of instructions 900. Non-transitory computer readable media includes all computer readable media, with the sole exception being a transitory, propagating signal per se. The instructions 900 are further operable to be transmitted or received over the network 810 via the network interface unit 896 as communication media, which is operable to include a modulated data signal such as a carrier wave or other transport mechanism and includes any delivery media. The term “modulated data signal” means a signal that has one or more of its characteristics changed or set in a manner as to encode information in the signal.

Storage devices 890 and memory 862 include, but are not limited to, volatile and non-volatile media such as cache, RAM, ROM, EPROM, EEPROM, FLASH memory, or other solid state memory technology; discs (e.g., digital versatile discs (DVD), HD-DVD, BLU-RAY, compact disc (CD), or CD-ROM) or other optical storage; magnetic cassettes, magnetic tape, magnetic disk storage, floppy disks, or other magnetic storage devices; or any other medium that can be used to store the computer readable instructions and which can be accessed by the computer system 800.

In one embodiment, the computer system 800 is within a cloud-based network. In one embodiment, the server 850 is a designated physical server for distributed computing devices 820, 830, and 840. In one embodiment, the server 850 is a cloud-based server platform. In one embodiment, the cloud-based server platform hosts serverless functions for distributed computing devices 820, 830, and 840.

In another embodiment, the computer system 800 is within an edge computing network. The server 850 is an edge server, and the database 870 is an edge database. The edge server 850 and the edge database 870 are part of an edge computing platform. In one embodiment, the edge server 850 and the edge database 870 are designated to distributed computing devices 820, 830, and 840. In one embodiment, the edge server 850 and the edge database 870 are not designated for distributed computing devices 820, 830, and 840. The distributed computing devices 820, 830, and 840 connect to an edge server in the edge computing network based on proximity, availability, latency, bandwidth, and/or other factors.

It is also contemplated that the computer system 800 is operable to not include all of the components shown in FIG. 12, is operable to include other components that are not explicitly shown in FIG. 12, or is operable to utilize an architecture completely different than that shown in FIG. 12. The various illustrative logical blocks, modules, elements, circuits, and algorithms described in connection with the embodiments disclosed herein are operable to be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application (e.g., arranged in a different order or partitioned in a different way), but such implementation decisions should not be interpreted as causing a departure from the scope of the present invention.

The above-mentioned examples are provided to serve the purpose of clarifying the aspects of the invention, and it will be apparent to one skilled in the art that they do not serve to limit the scope of the invention. By nature, this invention is highly adjustable, customizable and adaptable. The above-mentioned examples are just some of the many configurations that the mentioned components can take on. All modifications and improvements have been deleted herein for the sake of conciseness and readability but are properly within the scope of the present invention.

The invention claimed is:

1. A wearable device for health-monitoring, comprising:
an armband; and
at least one sensor attached to the armband;
wherein the at least one sensor is operable to gather physiological data regarding an individual;
wherein the at least one sensor is in network-based communication with at least one server, including a processor and a database; and
wherein the processor includes an artificial intelligence module operable to determine a current health state and/or a future health state of the individual based on the physiological data.

2. The wearable device of claim 1, wherein the current health state of the individual includes a COVID-19 infection.

3. The wearable device of claim 1, wherein the at least one sensor includes at least one body temperature sensor, at least one heart rate sensor, at least one pulse oximeter sensor, at least one blood pressure sensor, and/or at least one analyte sensor.

4. The wearable device of claim 1, wherein the network-based communication includes BLUETOOTH, WI-FI, and/or RFID-based communication.

5. The wearable device of claim 1, wherein the processor is operable to generate a user profile based on current and historical data generated by the at least one sensor.

6. The wearable device of claim 1, wherein the processor is operable to automatically recommend a treatment plan based on the physiological data and transmit the recommended treatment plan to a user device.

7. The wearable device of claim 1, wherein the at least one sensor is connected to a graphical user interface (GUI), and wherein the GUI is operable to display the physiological data.

8. The wearable device of claim 7, wherein the display of the physiological data includes at least one display bar graph, at least one line graph, at least one circle graph, at least one histogram, at least one mosaic chart, at least one spider chart, at least one flow chart, at least one control chart, at least one waterfall chart, at least one scatter plot, and/or at least one anatomical diagram.

9. The wearable device of claim 1, wherein the processor is operable to generate a medical alert, and transmit the medical alert to at least one third party device.

10. A method of health monitoring, comprising:
providing an armband including at least one sensor;
the at least one sensor generating physiological data regarding an individual;
the at least one sensor transmitting the physiological data to at least one server, including a processor and a database;
wherein the processor includes an artificial intelligence module; and
the artificial intelligence module determining a current health state and/or a future health state of the individual based on the physiological data.

11. The method of claim 10, wherein the current health state of the individual includes a COVID-19 infection.

12. The method of claim 10, wherein the at least one sensor includes at least one body temperature sensor, at least one heart rate sensor, at least one pulse oximeter sensor, at least one blood pressure sensor, and/or at least one analyte sensor.

13. The method of claim 10, further comprising the processor automatically recommending a treatment plan based on the physiological data and transmitting the recommended treatment plan to a user device.

14. The method of claim 10, wherein the at least one sensor is connected to a graphical user interface (GUI), further comprising the GUI displaying the physiological data.

15. The method of claim 14, wherein the display of the physiological data includes at least one display bar graph, at least one line graph, at least one circle graph, at least one histogram, at least one mosaic chart, at least one spider chart, at least one flow chart, at least one control chart, at least one waterfall chart, at least one scatter plot, and/or at least one anatomical diagram.

16. The method of claim 10, further comprising the processor generating a medical alert, and transmitting the medical alert to at least one third party device.

17. A system of health monitoring, comprising:
at least one sensor in network communication with at least one server, including a processor and a database; and
an armband, wherein the at least one sensor is attached to the armband;
wherein the at least one sensor is operable to generate physiological data and transmit the physiological data to the at least one server;
wherein the processor includes an artificial intelligence module operable to determine a current health state and/or a future health state of an individual based on the physiological data; and
wherein the processor is operable to automatically recommend a treatment plan based on the physiological data and transmit the recommended treatment plan to a user device.

18. The system of claim 17, wherein the current health state of the individual includes a COVID-19 infection.

19. The system of claim 17, wherein the at least one sensor includes at least one body temperature sensor, at least one heart rate sensor, at least one pulse oximeter sensor, at least one blood pressure sensor, and/or at least one analyte sensor.

20. The system of claim 17, wherein the at least one sensor is connected to a graphical user interface (GUI), and wherein the GUI is operable to display the physiological data.
